(19) United States
(12) Patent Application Publication
(10) Pub. No.: US 2023/0321155 A1
(43) Pub. Date: Oct. 12, 2023

(54) Use of Cell-Free Fat Extract for Treating Osteoporosis

(71) Applicant: SHANGHAI SEME CELL TECHNOLOGY CO., LTD., Shanghai (CN)

(72) Inventors: Zhifeng YU, Shanghai (CN); Wenjie ZHANG, Shanghai (CN)

(21) Appl. No.: 18/295,945

(22) Filed: Apr. 5, 2023

(30) Foreign Application Priority Data
Apr. 6, 2022 (CN) 202210359973.9

(51) Int. Cl. A61K 35/35; A61K 38/30; A61K 38/18; A61P 19/10

(52) U.S. Cl. CPC A61K 35/35; A61K 38/30; A61K 38/185; A61K 38/1825; A61K 38/1866; A61K 38/1841; A61K 38/1833; A61K 38/1858; A61P 19/10

(57) ABSTRACT

A cell-free fat extract may be used for treating osteoporosis. Specifically, the cell-free fat extract can be used to prepare a composition or preparation for preventing and/or treating osteoporosis, in particular osteoporosis due to estrogenic decline. The cell-free fat extract inhibits osteoblast differentiation and osteoclast differentiation, inhibits fusion of the bone marrow macrophage skeleton, improves the structure of hind limb bone, alleviates bone loss and attenuate bone resorption, and improves the mechanical properties of the femur.

### TECHNICAL FIELD

The present invention relates to the field of medicine, in particular, relates to the use of cell-free fat extract for treating osteoporosis.

### BACKGROUND TECHNIQUE

Postmenopausal osteoporosis is a systemic bone disease that is commonly seen in postmenopausal women due to a decrease in bone mineral density and bone mass caused by estrogen decline, and the destruction of bone microarchitecture, resulting in increased bone fragility and thus susceptibility to fracture.

The main principles of accurate osteoporosis treatment medications include calcium supplementation or increasing calcium absorption levels, promoting osteogenesis or inhibiting the resorption of osteoclasts.

Currently, traditional osteoporosis is treated mainly with medications, commonly used drugs include vitamin D, calcium, bisphosphonates, raloxifene, denosumab, teriparatide, abaloparatide and romosozumab. However, all of the above drugs have certain limitations and even side effects.
1. Bisphosphonates are synthetic pyrophosphate analogues, which can bind firmly to hydroxyapatite and inhibit bone resorption by deactivating osteoclasts. The most commonly used oral bisphosphonate is alendronate.
2. Raloxifene is a selective estrogen receptor modulator that can exert anti-resorption of estrogen on bone without deleterious effects on breast tissue. However, there is no evidence showing that raloxifene can prevent hip or non-vertebral fractures, in addition, raloxifene can cause adverse effects such as leg edema, cramps, hot flashes and venous thromboembolism.
3. Denosumab is a fully humanized antibody against receptor activator of nuclear factor-KB ligand (RANKL), a novel anti-resorptive agent. It may increase bone mineral density (BMD) levels and reduce the risk of vertebral, nonvertebral, and hip fractures in patients, but may produce adverse skin infections.
4. Teriparatide is a synthetic amino acid 1-34 fragment of human parathyroid hormone (PTH), can increase BMD levels, but may produce adverse effects such as nausea, headache, and dizziness.
5. Romosozumab is a humanized antibody that binds sclerostin with high affinity and increases bone mineral density, but long-term drug treatment can cause adverse effects such as atypical femoral fractures in the subtrochanteric region of femur and femoral shaft and osteonecrosis of the jaw.

Therefore, there is a need in this field to develop a drug that can effectively treat osteoporosis with high safety and low side effects.

### SUMMARY OF THE INVENTION

The object of the present invention is to provide a use of cell-free fat extract for preventing and/or treating osteoporosis.

In the first aspect of the present invention, it provides a use of a cell-free fat extract for the preparation of a composition or preparation, the composition or preparation is used for preventing and/or treating osteoporosis.

In another preferred embodiment, the osteoporosis is postmenopausal osteoporosis.

In another preferred embodiment, the osteoporosis is the osteoporosis caused by estrogenic decline.

In another preferred embodiment, the estrogen decline refers to 20%, 30%, 40%, 50%, 60%, 70%, 80%, or 90% decrease in estrogen levels compared to when there was no decline.

In another preferred embodiment, the prevention and/or treatment of osteoporosis comprises one or more features selected from the group consisting of:
(i) inhibiting osteoblast differentiation and osteoclast differentiation;
(ii) inhibiting fusion of the BMM cytoskeleton;
(iii) increasing bone volume fraction;
(iv) increasing the number of bone trabecula;
(v) increasing bone connection density;
(vi) decreasing trabecular separation.

In another preferred embodiment, the cell-free fat extract is derived from adipose tissue, preferably from allogeneic adipose tissue, more preferably from human adipose tissue.

In another preferred embodiment, the cell-free fat extract is a cell-free fat extract obtained by extraction and preparation from fat in human or non-human mammals.

In another preferred embodiment, the non-human mammal is a monkey, an orangutan, a cow, a pig, a dog, a sheep, a rat or a rabbit.

In another preferred embodiment, the composition or preparation comprises pharmaceutical composition or preparation, food composition or preparation, nutraceutical composition or preparation, or dietary supplement.

In another preferred embodiment, the composition or preparation further comprises pharmaceutically, food, nutraceutical, or dietary acceptable carriers.

In another preferred embodiment, the dosage form of the composition or preparation is a solid dosage form, a semi-solid dosage form, or a liquid dosage form.

In another preferred embodiment, the dosage form of the composition or preparation is oral preparation, external preparation, or injectable preparation.

In another preferred embodiment, the injectable preparation is an intravenous injection or an intramuscular injection.

In another preferred embodiment, the dosage form of the composition or preparation is powder, granule, capsule, injection, tincture, oral solution, tablet or lozenge.

In another preferred embodiment, the composition or preparation further comprises other drugs for preventing and/or treating osteoporosis.

In another preferred embodiment, the other drugs for preventing and/or treating osteoporosis are selected from the group consisting of vitamin D, calcium agents, bisphosphonates, raloxifene, denosumab, teriparatide, abaloparatide, and romosozumab, and the combinations thereof.

In another preferred embodiment, the composition or preparation is administered externally, topically, or by injection.

In another preferred embodiment, the composition or preparation contains at least 1 wt %, at least 5 wt %, at least 20 wt %, at least 30 wt %, at least 40 wt %, at least 50 wt %, at least 60 wt %, at least 70 wt %, at least 80 wt %, or at least 90 wt %, preferably 95 wt %, more preferably, 98 wt %, most preferably 99 wt % of the cell-free fat extract, by total weight of the composition or product.

In another preferred embodiment, the cell-free fat extract contains no cell and no lipid droplet.

In another preferred embodiment, the lipid droplets are oil droplets released after fat cells are disrupted.

In another preferred embodiment, the expression of “contain no lipid droplet” means that in the cell-free fat extract, the volume of oil droplets accounts for less than 1%, preferably less than 0.5%, more preferably less than 0.1% of the total liquid.

In another preferred embodiment, the cells are selected from the group consisting of endothelial cells, adipose stem cells, macrophages, and stromal cells.

In another preferred embodiment, the expression of “contain no cell” means that the average number of cells in 1 mL of the cell-free fat extract is ≤1, preferably ≤0.5, more preferably ≤0.1, or 0.

In another preferred embodiment, the cell-free fat extract is a naturally-obtained nano-fat extract without added ingredients.

In another preferred embodiment, the expression of “without added ingredients” means that no solution, solvent, small molecule, chemical preparation, and biological additive are added during the preparation of the fat extract except rinsing step.

In another preferred embodiment, the fat extract is not stromal vascular fraction (SVF).

In another preferred embodiment, the cell-free fat extract is prepared by centrifuging the adipose tissue after emulsification.

In another preferred embodiment, the cell-free fat extract contains one or more components selected from the group consisting of IGF-1, BDNF, GDNF, TGF-β, HGF, bFGF, VEGF, PDGF, EGF, NT-3, GH, G-CSF, and the combinations thereof.

In another preferred embodiment, in the cell-free fat extract, the concentration of IGF-1 is 5000-30000 pg/ml, preferably 6000-20000 pg/ml, more preferably 7000-15000 pg/ml, more preferably 8000-12000 pg/ml, more preferably 9000-11000 pg/ml, more preferably 9500-10500 pg/ml.

In another preferred embodiment, in the cell-free fat extract, the concentration of BDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1850 pg/ml.

In another preferred embodiment, in the cell-free fat extract, the concentration of GDNF is 800-5000 pg/ml, preferably 1000-4000 pg/ml, more preferably 1200-2500 pg/ml, more preferably 1400-2000 pg/ml, more preferably 1600-2000 pg/ml, more preferably 1700-1900 pg/ml.

In another preferred embodiment, in the cell-free fat extract, the concentration of bFGF is 50-600 pg/ml, preferably 100-500 pg/ml, more preferably 120-400 pg/ml, more preferably 150-300 pg/ml, more preferably 200-280 pg/ml, more preferably 220-260 pg/ml.

In another preferred embodiment, in the cell-free fat extract, the concentration of VEGF is 50-500 pg/ml, preferably 100-400 pg/ml, more preferably 120-300 pg/ml, more preferably 150-250 pg/ml, more preferably 170-230 pg/ml, more preferably 190-210 pg/ml.

In another preferred embodiment, in the cell-free fat extract, the concentration of TGF-β is 200-3000 pg/ml, preferably 400-2000 pg/ml, more preferably 600-1500 pg/ml, more preferably 800-1200 pg/ml, more preferably 800-1100 pg/ml, more preferably 900-1000 pg/ml.

In another preferred embodiment, in the cell-free fat extract, the concentration of HGF is 200-3000 pg/ml, preferably 400-2000 pg/ml, more preferably 600-1500 pg/ml, more preferably 600-1200 pg/ml, more preferably 800-1000 pg/ml, more preferably 850-950 pg/ml.

In another preferred embodiment, in the cell-free fat extract, the concentration of PDGF is 50-600 pg/ml, preferably 80-400 pg/ml, more preferably 100-300 pg/ml, more preferably 140-220 pg/ml, more preferably 160-200 pg/ml, more preferably 170-190 pg/ml.

In another preferred embodiment, the weight ratio of IGF-1 to VEGF is 20-100:1, preferably 30-70:1, more preferably 40-60:1, and most preferably 45-55:1.

In another preferred embodiment, the weight ratio of BDNF to VEGF is 2-20:1, preferably 4-15:1, more preferably 6-12:1, and most preferably 8-9.5:1.

In another preferred embodiment, the weight ratio of GDNF to VEGF is 2-20:1, preferably 4-15:1, more preferably 6-12:1, and most preferably 8.5-9.5:1.

In another preferred embodiment, the weight ratio of bFGF to VEGF is 0.2-8:1, preferably 0.5-5:1, more preferably 0.6-2:1, more preferably 0.8-1.6:1, and most preferably 1-1.5:1.

In another preferred embodiment, the weight ratio of TGF-β to VEGF is 1-20:1, preferably 1-15:1, more preferably 1-10:1, more preferably 2-8:1, more preferably 4-6:1.

In another preferred embodiment, the weight ratio of HGF to VEGF is 1-20:1, preferably 1-15:1, more preferably 1-10:1, more preferably 2-8:1, more preferably 4-5.5:1.

In another preferred embodiment, the weight ratio of PDGF to VEGF is 0.1-3:1, preferably 0.2-2:1, more preferably 0.4-1.5:1, and most preferably 0.7-1.2:1.

In another preferred embodiment, the cell-free fat extract is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the liquid at the bottom layer and the oil at the upper layer from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

In another preferred embodiment, in the step (2), the centrifugation is performed at 800-2500 g, preferably 800-2000 g, more preferably 1000-1500 g, and most preferably 1100-1300 g.

In another preferred embodiment, in the step (2), the centrifugation time is 1-15 min, preferably 1-10 min, more preferably 1-8 min, and most preferably 1-5 min.

In another preferred embodiment, the centrifugation temperature is 2-6° C.

In another preferred embodiment, in the step (4), the emulsification is mechanical emulsification.

In another preferred embodiment, the mechanical emulsification is performed by repeated blowing by a syringe (e. g., blowing 20-200 times, preferably 20-150 times, more preferably 20-100 times, more preferably 40-70 times).

In another preferred embodiment, the blowing method is that two 20 ml injection syringes are connected to a Luer-lock connector and repeatedly push at a constant speed.

In another preferred embodiment, in the step (4), the emulsification is by means of crushing through a tissue homogenizer.

In another preferred embodiment, the step (5) further includes freezing and thawing the emulsified fat mixture before centrifuging the emulsified fat mixture.

In another preferred embodiment, the thawed mixture is used for centrifugation after freezing and thawing treatment.

In another preferred embodiment, the freezing temperature is from −50° C. to −120° C., preferably from −60° C. to −100° C., more preferably from −70° C. to −90° C.

In another preferred embodiment, the thawing temperature is 20-40° C., preferably 25-40° C., more preferably 37° C.

In another preferred embodiment, the number of cycles of thawing after freezing is 1-5 (preferably 1, 2, 3 or 4).

In another preferred embodiment, in the step (5), after centrifugation, the emulsified fat mixture is layered into four layers, the first layer is an oil layer, the second layer is a residual fat tissue layer, the third layer is a liquid layer (i. e., an intermediate liquid layer), and the fourth layer is a cell debris particles at the bottom.

In another preferred embodiment, in the step (5), the centrifugation is performed at 800-2500 g, preferably 800-2000 g, more preferably 1000-1500 g, and most preferably 1100-1300 g.

In another preferred embodiment, in the step (5), the centrifugation time is 1-15 min, preferably 1-10 min, more preferably 2-8 min, and most preferably 3-7 min.

In another preferred embodiment, the centrifugation temperature is 2-6° C.

In another preferred embodiment, in the step (5), the first layer, the second layer, the third layer and the fourth layer are sequentially arranged from top to bottom.

In another preferred embodiment, in the step (5), the intermediate liquid layer is a transparent or substantially transparent layer.

In another preferred embodiment, in the step (6), the filtration can remove fat cells from the primary fat extract.

In another preferred embodiment, in the step (6), the filtering and sterilization are carried out through a filter (such as a 0.22 μm microporous filter membrane).

In another preferred embodiment, the filter is a microporous membrane filter.

In another preferred embodiment, the pore size of the microporous membrane is 0.05-0.8 μm, preferably 0.1-0.5 μm, more preferably 0.1-0.4 μm, more preferably 0.15-0.3 μm, more preferably 0.2-0.25 μm, and most preferably 0.22 μm.

In another preferred embodiment, in the step (6), the filtering and sterilization is carried out by first filtering through a first filter that can filter cells, and then through a second filter (such as a 0.22 μm filter) that can filter pathogens (such as bacteria).

In another preferred embodiment, the step (6) further includes subpackaging the fat extract to obtain a subpackaging product. (The subpacked extract can be stored at −20° C. for later use; it can be used directly after thawing at low temperature (e. g. −4° C.) or at normal temperature, or stored at low temperature (e. g. 4° C.) for a period of time for later use after thawing).

In the second aspect of the present invention, it provides a use of proteins in a cell-free fat extract for the preparation of a composition or preparation, the composition or preparation is used for preventing and/or treating osteoporosis.

In another preferred embodiment, the cell-free fat extract is as described in the first aspect of the invention.

In another preferred embodiment, the protein in the cell-free fat extract is a cell-free fat extract after RNA enzyme digestion.

In another preferred embodiment, the protein in the cell-free fat extract is obtained by optional extracting the cell-free fat extract after RNA enzyme digestion.

In the third aspect of the present invention, it provides a use of the cationic component in a cell-free fat extract for the preparation of a composition or preparation, the composition or preparation is used for preventing and/or treating osteoporosis.

In another preferred embodiment, the cell-free fat extract is as described in the first aspect of the present invention.

In another preferred embodiment, the cationic component in the cell-free fat extract is the cationic component obtained by ion chromatography of the cell-free fat extract.

In another preferred embodiment, the cationic component in the cell-free fat extract is obtained by optionally extracting the cationic component obtained by ion chromatography of the cell-free fat extract.

In the fourth aspect of the present invention, it provides a method for preventing and/or treating osteoporosis, comprising administering a cell-free fat extract to a subject in need thereof.

In another preferred embodiment, the subject is a human or non-human mammal.

In another preferred embodiment, the non-human mammal comprises a rodent, such as a rat, a mouse.

In another preferred embodiment, the administration is by oral administration, topical administration or injection administration, preferably intravenous administration.

It should be understood that, within the scope of the present invention, the above technical features of the present invention and the technical features specifically described in the following descriptions (such as the examples) can be combined with each other to form a new or preferred technical solution. Due to space limitations, they will not be repeated herein.

### DESCRIPTION OF THE DRAWINGS

FIG. 1 shows the process of preparing the cell-free fat extract of the present invention.

FIG. 2 shows the results of CCK-8 assay for BMM cell viability.

FIG. 3 shows the effect of CEFFE on the expression of mRNA related to osteoblast differentiation and osteoclast differentiation in mice BMM cells detected by QPCR.

FIG. 4 shows the effect of CEFFE on osteoblast differentiation and osteoclast differentiation in mice BMM cells detected by TRAP staining.

FIG. 5 shows the effect of CEFFE group and control group on cell fusion during osteoblast differentiation and osteoclast differentiation of mice BMM cell observed by immunofluorescence staining of phalloidin.

FIG. 6 shows the rt-PCR results of extracted RNA of mice BMM cell after adding protease to digest CEFFE and adding RNAase to digest CEFFE.

FIG. 7 shows the effect of anionic component, cationic component and strong cationic component of CEFFE on RNA-related expression level in mice BMM cells.

FIG. 8 shows the effect of different charge components contained in CEFFE on the osteoblast differentiation and osteoclast differentiation of BMM detected by TRAP staining.

FIG. 9 shows the therapeutic effect of CEFFE on bone loss in the hind limbs of tail suspension mice.

FIG. 10 shows the effect of CEFFE on bone mass changes in tibia and mechanical properties of femur.

### DETAILED DESCRIPTION OF THE INVENTION

After extensive and thorough research, the present inventors have developed a cell-free fat extract with excellent therapeutic effects for osteoporosis (especially osteoporosis due to estrogen decline) for the first time. Because the extract of the present invention is a cell-free liquid component and easy to prepare, its use can theoretically avoid cell-related problems in clinical applications with high safety and low side effects. The present invention has been completed on this basis.

Terms Unless otherwise defined, all technical and scientific terms used herein have the same meaning as commonly understood by one of ordinary skill in the art to which this invention belongs.

As used herein, the terms “include” “contain” and “comprise” are used interchangeably and include not only open-ended definition, but also semi-closed definition, and closed definition. In other words, the terms include “consisting of”, “substantially consisting of”.

As used herein, “IGF-1” refers to insulin-like growth factors-1.

As used herein, “BDNF” refers to brain-derived neurotrophic factor.

As used herein, “GDNF” refers to glial cellline-derived neurotrophic factor.

As used herein, “bFGF” refers to basic fibroblast growth factor.

As used herein, “VEGF” refers to vascular endothelial growth factor.

As used herein, “TGF-β” refers to transforming growth factor-β1.

As used herein, “HGF” refers to hepatocyte growth factor.

As used herein, “PDGF” refers to platelet derived growth factor.

As used herein, “EGF” refers to epidermal growth factor.

As used herein, “NT-3” refers to neurotrophins-3.

As used herein, “GH” refers to growth hormone.

As used herein, “G-CSF” refers to granulocyte colony stimulating factor.

Cell Free Fat Extract (CEFFE) and the Preparation Method Therefor

As used herein, the term of “cell free fat extract of the present invention” refers to an adipose tissue-derived extract (or extract liquid) prepared without adding any solution, solvent, small molecule, chemical preparation, and biological additive during the preparation of the fat extract (except for the rinsing step).

Preferably, the cell-free fat tissue extract is extracted from allogeneic adipose tissue, such as human.

In addition, it should be understood that although it is not necessary to add any additives (or added ingredients) during the preparation process of the extract of the present invention, some or small amount of safe substance (such as small amount of water) that do not negatively or adversely affect the activity of the extract herein may also be added.

The cell-free fat extract of the present invention may be derived from human adipose tissue, which is purified from nano-fat by removing the oil and cellular/extracellular matrix fraction after centrifugation. It is a cell-free liquid rich in various growth factors and is easy to prepare. We have found that the extract contains cytokines and growth factors such as BDNF, GDNF, TGF-β, HGF, bFGF, VEGF, PDGF, EGF, NT-3 and G-CSF.

Representatively, the cell-free fat extract described in the present invention is prepared by the following method:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the liquid layer at the bottom and the oil layer on top from the layered mixture and collecting the intermediate layer (that is, the fat layer containing fat cells);
(4) subjecting the intermediate layer to emulsification to obtain a emulsified fat mixture (also called nano-fat);
(5) centrifuging the emulsified fat mixture to obtain an intermediate liquid layer, which is a primary fat extract; and
(6) subjecting the primary fat extract to filtration and sterilization to obtain the cell-free fat extract.

Use

The present invention provides a use of a cell-free fat extract for the preparation of composition or preparation, the composition or preparation is used for preventing and/or treating osteoporosis.

Osteoporosis is classified as primary osteoporosis and secondary osteoporosis.

Primary osteoporosis is further classified into postmenopausal osteoporosis (type I), senile osteoporosis (type II) and idiopathic osteoporosis (including adolescent type).

In addition to primary osteoporosis mainly associated with menopause and old age, osteoporosis may also be caused by a variety of diseases, called secondary osteoporosis. Common diseases that may cause osteoporosis include endocrine diseases, including diabetes mellitus (type 1 and type 2), hyperparathyroidism, Cushing syndrome, hypogonadism, hyperthyroidism, pituitary prolactinoma, hypopituitarism; connective tissue diseases, including systemic lupus erythematosus, rheumatoid arthritis, dry syndrome, dermatomyositis, mixed connective tissue diseases; chronic kidney diseases, including renal osteodystrophy; gastrointestinal diseases and nutritional disorders, including malabsorption syndrome, post-gastrectomy, chronic pancreatic disease, chronic liver disorders, malnutrition, long-term intravenous nutritional support therapy; hematologic diseases, including leukemia, lymphoma, multiple myeloma, Gaucher disease, and myelodysplastic syndromes; neuromuscular system diseases, including hemiplegia from all causes, paraplegia, motor dysfunction, muscular dystrophy, stiff-man syndrome and myotonicmyopathies; after organ transplantation; long-term use of the following drugs, said drugs include glucocorticoids, immunosuppressants, heparin, anticonvulsants, anticancer drugs, aluminum-containing antacids, thyroid hormones, chronic fluorosis, gonadotropin-releasing hormone analogues (GnRHa) or dialysis solution for renal failure, or similar causes.

In the present invention, the term “prevent” refers to a method of preventing the onset of a disease and/or its attendant symptoms or protecting a subject from the disease. As used herein, “prevent” also includes delaying the onset of a disease and/or its attendant symptoms and reducing the subject's risk for the disease.

“Treat” as used herein includes delaying and terminating the progression of a disease, or eliminating a disease, and does not require suppression, elimination, or reversal with 100% percentage. In some embodiments, the cell-free fat extract described herein reduces, inhibits, and/or reverses osteoporosis by, for example, at least about 10%, at least about 30%, at least about 50%, or at least about 80%, compared to the absence of the cell-free fat extract described herein.

The present invention also provides a method for preventing and/or treating osteoporosis by administering a cell-free fat extract as described herein to a subject in need thereof.

In another preferred embodiment, the subject is a human or non-human mammal.

In another preferred embodiment, the non-human mammal comprises a rodent, such as a rat, a mouse.

In another preferred embodiment, the administration is by oral administration, topical administration or injection administration.

Compositions and Administration

The compositions described herein include (but are not limited to) pharmaceutical compositions, food compositions, health care compositions, and dietary supplements, etc.

Representatively, the cell-free fat extract of the present invention may be prepared as pharmaceutical compositions in the dosage forms such as tablet, capsule, powder, micronized formulation, solution, lozenge, jelly, cream formulations, spirit, suspension, tincture, poultice, liniment, lotion, and aerosol. Pharmaceutical compositions can be prepared by commonly known preparation techniques, and suitable pharmaceutical additives can be added to the drug.

The compositions of the present invention may also include pharmaceutically, food, nutraceutical, or dietary acceptable carriers. “Pharmaceutically, food, nutraceutical or dietary acceptable carrier” means one or more compatible solids or liquid fillers, or gelatinous materials which are suitable for human use and should be of sufficient purity and sufficiently low toxicity. “Compatible” means that each component in the composition can be admixed with the compounds of the present invention and with each other without significantly reducing the efficacy of the compounds. Some examples of pharmaceutically, food, nutraceutical or dietary acceptable carriers include cellulose and the derivatives thereof (such as sodium carboxymethyl cellulose, sodium ethyl cellulose, cellulose acetate, etc.), gelatin, talc, solid lubricants (such as stearic acid, magnesium stearate), calcium sulfate, vegetable oils (such as soybean oil, sesame oil, peanut oil, olive oil, etc.), polyols (such as propylene glycol, glycerol, mannitol, sorbitol, etc.), emulsifiers (such as Tween®), wetting agent (such as sodium dodecyl sulfate), coloring agents, flavoring agents, stabilizers, antioxidants, preservatives, pyrogen-free water, etc.

There is no special limitation of administration mode for the compositions of the present invention, and the representative administration mode includes (but is not limited to) oral administration, parenteral (intravenous or intramuscular) administration, or topical administration, preferably oral administration and injection administration.

The dosage form of the composition or preparation described herein is oral preparation, external preparation, or injection preparation. Representatively, solid dosage forms for oral administration include capsules, tablets, pills, powders and granules. In these solid dosage forms, the active compounds are mixed with at least one conventional inert excipient (or carrier), such as sodium citrate or CaHPO₄, or mixed with any of the following components: (a) fillers or compatibilizer, for example, starch, lactose, sucrose, glucose, mannitol and silicic acid; (b) binders, for example, hydroxymethyl cellulose, alginates, gelatin, polyvinylpyrrolidone, sucrose and arabic gum; (c) humectant, such as, glycerol; (d) disintegrating agents such as agar, calcium carbonate, potato starch or tapioca starch, alginic acid, certain composite silicates, and sodium carbonate; (e) dissolution-retarding agents, such as paraffin; (f) absorption accelerators, for example, quaternary ammonium compounds; (g) wetting agents, such as cetyl alcohol and glyceryl monostearate; (h) adsorbents, for example, kaolin; and (i) lubricants such as talc, calcium stearate, magnesium stearate, solid polyethylene glycol, sodium lauryl sulfate, or the mixtures thereof. In capsules, tablets and pills, the dosage forms may also contain buffering agents.

The solid dosage forms such as tablets, sugar pills, capsules, pills and granules can be prepared by using coating and shell materials, such as enteric coatings and any other materials known in the art. They can contain an opaque agent.

Liquid dosage forms for oral administration include pharmaceutically acceptable emulsions, solutions, suspensions, syrups or tinctures. In addition to the active compounds, the liquid dosage forms may contain any conventional inert diluents known in the art such as water or other solvents, solubilizers and emulsifiers, for example, ethanol, isopropanol, ethyl carbonate, ethyl acetate, propylene glycol, 1,3-butanediol, dimethyl formamide, as well as oil, in particular, cottonseed oil, peanut oil, corn germ oil, olive oil, castor oil and sesame oil, or the combinations thereof.

Besides these inert diluents, the composition may also contain additives such as wetting agents, emulsifiers, and suspending agent, sweetener, flavoring agent and perfume.

In addition to the active component, the suspension may contain suspending agent, for example, ethoxylated isooctadecanol, polyoxyethylene sorbitol and sorbitan esters, microcrystalline cellulose, methanol aluminum and agar, or the combinations thereof.

The compositions for parenteral injection may comprise physiologically acceptable sterile aqueous or anhydrous solutions, dispersions, suspensions or emulsions, and sterile powders which can be re-dissolved into sterile injectable solutions or dispersions. Suitable aqueous and non-aqueous carriers, diluents, solvents or excipients include water, ethanol, polyols and any suitable mixtures thereof.

The dosage forms of the compounds of the invention for topical administration include ointment, powder, patch, spray, and inhalant. The active ingredient is mixed under sterile conditions with physiologically acceptable carriers and any preservatives, buffers, or propellants that may be required if necessary.

The cell-free fat extract of the present invention can be administered alone or in combination with other drugs for preventing and/or treating osteoporosis. Wherein, the drugs that can be co-administered include, but are not limited to vitamin D, calcium agents, bisphosphonates, raloxifene, denosumab, teriparatide, abaloparatide, and romosozumab.

When the pharmaceutical compositions are administered, a safe and effective amount of cell free fat extract of the present invention is administered to a human or non-human mammal (such as rat, mouse, dog, cat, cow, chicken, duck, etc.) in need thereof, wherein the dose of administration is an effective administration dosage which is considered pharmaceutically, food or nutraceutical acceptable. As used herein, the term “safe and effective amount” refers to an amount that is functional or active in humans and/or animals and is acceptable to humans and/or animals. It should be understood by those of ordinary skill in the art that the “safe and effective amount” may vary depending on the form of the pharmaceutical composition, the route of administration, the excipients of the drug used, the severity of the disease, and the combination with other drugs. For example, for a person weighed 60 kg, the daily dose is usually 0.1 to 1000 mg, preferably 1 to 600 mg, more preferably 2 to 300 mg. Of course, the specific dose should also take into account the route of administration, patient healthy status and other factors, which are within the skill of an experienced physician.

The Main Advantages of the Present Invention Include:
1. The present invention discover that the cell-free fat extract have excellent preventive and/or therapeutic effect on osteoporosis for the first time.
2. The cell-free fat extract of the present invention can inhibit osteoblast differentiation and osteoclast differentiation at low concentrations, which is also dose-dependent.
3. The cell-free fat extract of the present invention can inhibit the fusion of BMM cytoskeleton and inhibit the differentiation of BMM to mature multinucleated osteoclasts.
4. The cell-free fat extract of the present invention can improve the structure of hind limb bone, alleviate bone loss and attenuate bone resorption.
5. The cell-free fat extract described in the present invention is a cell-free component that can avoid cell-related problems in clinical applications, such as genetic stability of cells after processing, cell activity and survival after injection, multiple administration storage of cells, and immunogenicity of cells when using allogeneic fat, and the cell-free fat extract described in the present invention has advantages of high safety and lower side effects in the prevention and treatment of osteoporosis.

The present invention will be further illustrated below with reference to the specific examples. It should be understood that these examples are only to illustrate the invention but not to limit the scope of the invention. The experimental methods with no specific conditions described in the following examples are generally performed under the conventional conditions, or according to the manufacturer's instructions. Unless indicated otherwise, percentage and parts are calculated by weight.

### Example 1. Preparation of Cell-Free Fat Extract (CEFFE)

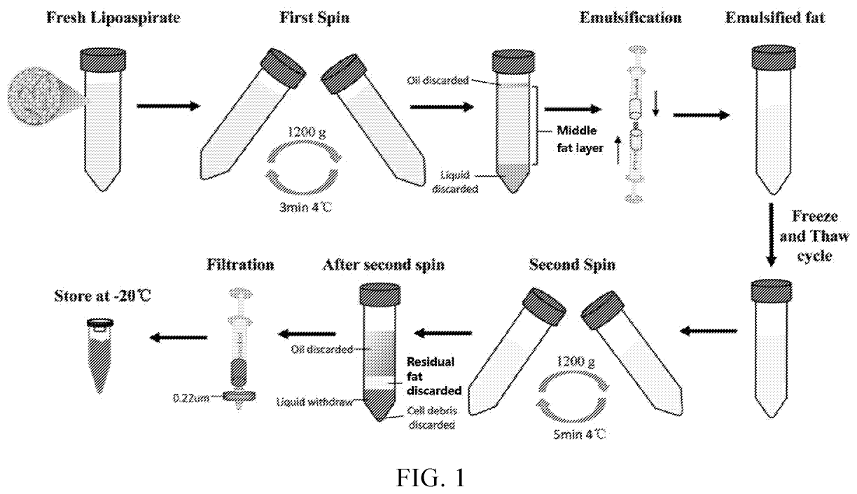

The fat was obtained from volunteers with informed consent. The flow chart for the preparation of cell-free fat extract is shown in FIG. 1, with the following steps:

The detailed steps for isolation of CEFFE are shown in FIG. 1. The lipoaspirate was first rinsed with saline to remove erythrocytes and then centrifuged at 1200×g for 3 min. After the first centrifugation, the oil at the upper layer and the liquid at the bottom layer were discarded and the middle fat layer was collected for mechanical emulsification. Emulsified fat was obtained by moving back and forth for 60 times in two 20 ml syringes connected by a Luer-lock connector with an inner diameter of 2 mm (B. Braun Medical Inc., Melsungen, Germany). After the emulsified fat was further centrifugation at 1200×g for 5 min, the fat was separated into 4 layers. The oil at the upper layer, the fat at the second layer and the cell debris pellet at the bottom were discarded. The slightly pinkish aqueous CEFFE at the third layer was collected, filtered through a 0.22 μm filter and stored at −20° C. for later experiments.

The cytokine content of the prepared cell-free fat extract was measured using ELISA immunosorbent assay kits, including cytokines such as IGF-1, BDNF, GDNF, bFGF, VEGF, TGF-β1, HGF and PDGF. The average concentrations of the six samples tested were as follows: IGF-1 (9840.6 pg/ml), BDNF (1764.5 pg/ml), GDNF (1831.9 pg/ml), bFGF (242.3 pg/ml), VEGF (202.9 pg/ml), TGF-β1 (954.5 pg/ml), HGF (898.4 pg/ml), and PDGF (179.9 pg/ml).

### Example 2. Experimental Method

The following technical protocol was used in the present invention: mice ovariectomy (OVX) model was established, and the changes in bone mass in the hind limbs of mice were observed after tail vein injection administration of CEFFE. The specific methods are as follows:

Mice OVX model: 8-week-old C57 female mice were used, and the mice were randomly divided into 3 groups: sham-operated control group (sham, n=10), PBS-treated OVX group (OVX, n=10), and CEFFE-treated OVX group (OVX+CEFFE, n=10). Anesthesia was induced using 1.5% isoflurane (IsoFlo, Abbott Laboratories) at a rate of 0.5-1.0 L/min. After shaving the hair in dorsal mid-lumbar region, the skin was cleaned and a 20 mm midline dorsal skin incision was made. After excision of a pair of ovaries, the wound was closed by suturing. The same procedure was performed in sham-operated control mice but without ovary removal. The drug was administered 8 weeks after modeling.

### Example 3. Effect of CEFFE on Bone Marrow-Derived Macrophages (BMM Cells) in Mice

3.1 Effect of CEFFE on the Proliferation of Mouse BMM Cells

Mouse-derived BMM cells were used and inoculated in two 96-well plates, after 12 h, cell attachment were completed. DMEM medium containing different concentrations of CEFFE (25, 50, 100, 250 and 500 μg/mL) was added during the fluid change, and after 24 h and 48 h, respectively, the fluid in well plates were changed to DMEM medium containing 10% CCK-8 reagent and placed in a 37 degree incubator. After 2 h, the absorbance at 450 nm was detected using a microplate reader (650 nm was chosen as a reference).

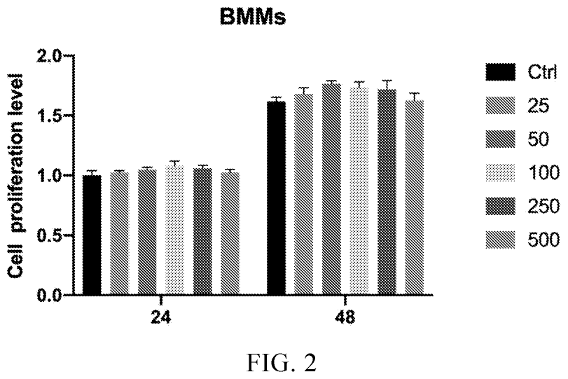

The results of CCK-8 assay for cell viability were shown in FIG. 2, which showed that CEFFE has no significant effect on proliferation of BMM cells in a certain concentration range (25, 50, 100, 250 and 500 μg/mL).

3.2 Effect of CEFFE on Osteoblast Differentiation and Osteoclast Differentiation of Mice BMM Cells

Mouse-derived BMM cells were used and inoculated in 12-well plates and 24-well plates, respectively, and cell attachment were completed after 12 h. Osteoclast-inducing medium containing different concentrations of CEFFE (25, 50, 100, 250 and 500 μg/mL) was added during the fluid change, and the medium was replaced with new CEFFE-containing medium every two days. mRNAs of cells in each group were extracted and TRAP staining was performed after six days.

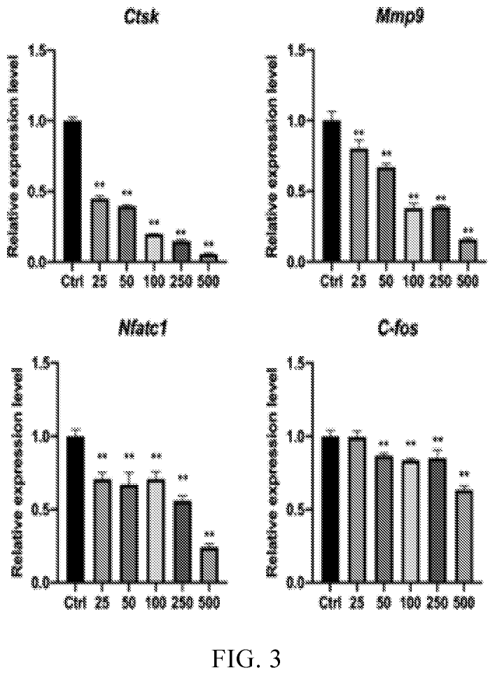

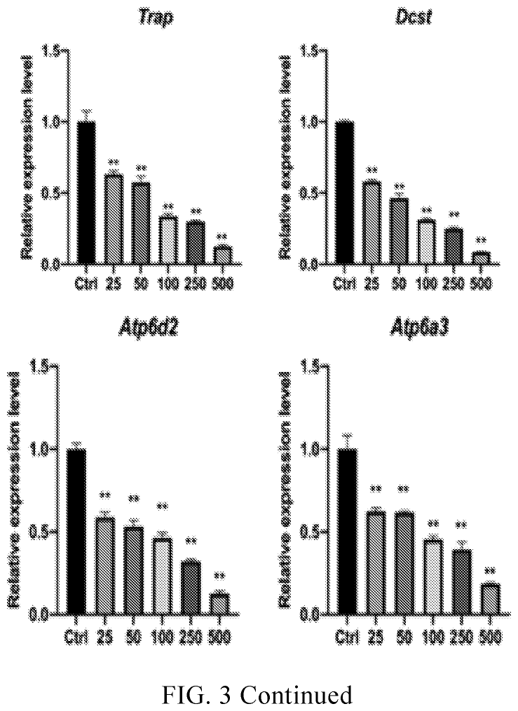

FIG. 3 showed the effect of CEFFE on the expression of mRNA related to osteoblast differentiation and osteoclast differentiation of BMM detected by QPCR, which showed that CEFFE could inhibit osteoblast differentiation and osteoclast differentiation of mice BMM cells at the concentration of 0.5%.

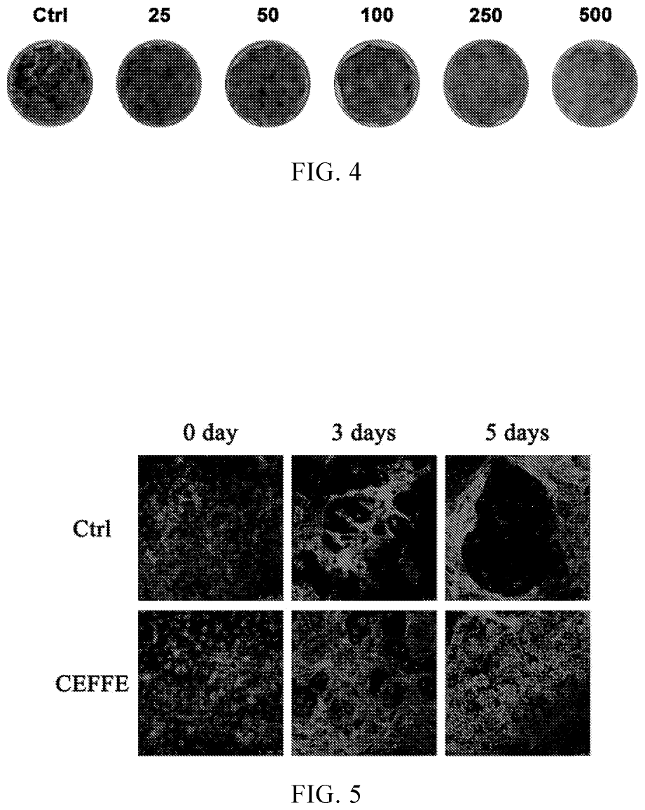

FIG. 4 showed the effect of CEFFE on the osteoblast differentiation and osteoclast differentiation of BMM cells detected by TRAP staining. The control group (Ctrl) showed positive TRAP staining after six days of induction by adding RANKL, however, after adding RANKL and different concentrations of CEFFE (25, 50, 100, 250, 500 μg/mL) simultaneously, it showed a concentration gradient inhibitory effect of osteoclast differentiation of BMM, so the concentration of 50 μg/mL was chosen to detect the inhibitory effect of CEFFE on osteoclast differentiation based on this result.

3.3 Effect of CEFFE on Cell Fusion During Osteoblast Differentiation and Osteoclast Differentiation of Mice BMM Cells

Mouse-derived BMM cells were used and inoculated in 6 confocal dishes, and after cell attachment, the 6 confocal dishes were divided into two groups: control group (Ctrl) and CEFFE group, osteoclast induction medium was added to control group, and osteoclast medium containing 50 μg/mL CEFFE was added to CEFFE group. One dish from each group was fixed on days 0, 3 and 5, respectively, and immunofluorescence staining of phalloidin was performed. The cell fusion of each group was observed, and the results are shown in FIG. 5.

According to FIG. 5, 50 μg/mL of CEFFE significantly inhibited the fusion of BMM cytoskeleton and the differentiation of BMM to mature multinucleated osteoblasts on days 3 and 5 of BMM differentiation.

### Example 4. Study of the Main Components of CEFFE that Inhibit Osteoblast Differentiation and Osteoclast Differentiation of BMM

4.1 Proteins in CEFFE are the Main Component that Inhibit Osteoblast Differentiation and Osteoclast Differentiation of BMM

Mouse-derived BMM cells were used and inoculated in six 12-well plates, and the cells were divided into six groups after cell attachment, wherein osteoclast differentiation induction solution was added to the Ctrl group, osteoclast differentiation induction solution containing 50 μg/mL of CEFFE was added to CEFFE group, osteoclast differentiation induction solution containing CEFFE which was previously digested with 0.5 μg/mL or 1 μg/mL of Proteinase K was added to groups 3 and 4, and osteoclast differentiation medium containing CEFFE which was previously digested with 30 μg/mL or 50 μg/mL of RNase was added to groups 5 and 6. The medium was replaced with new medium every two days, and mRNAs of cells in each group were extracted after six days.

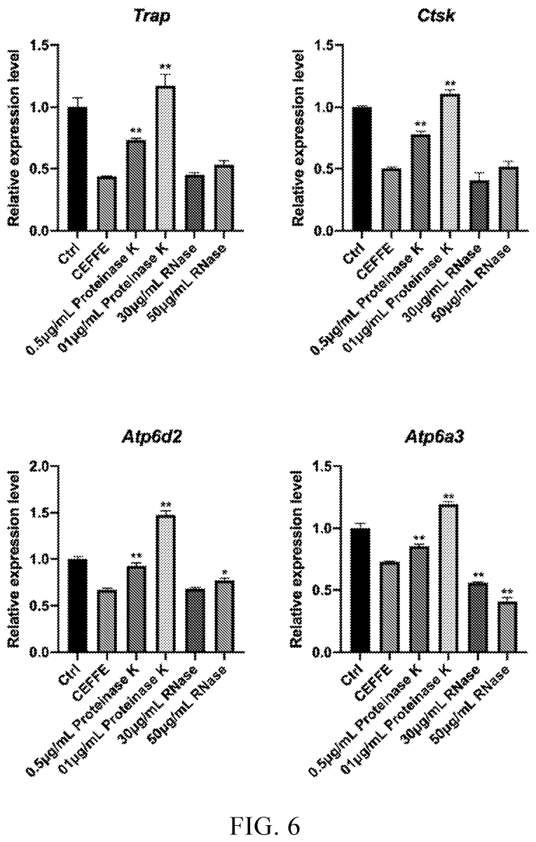

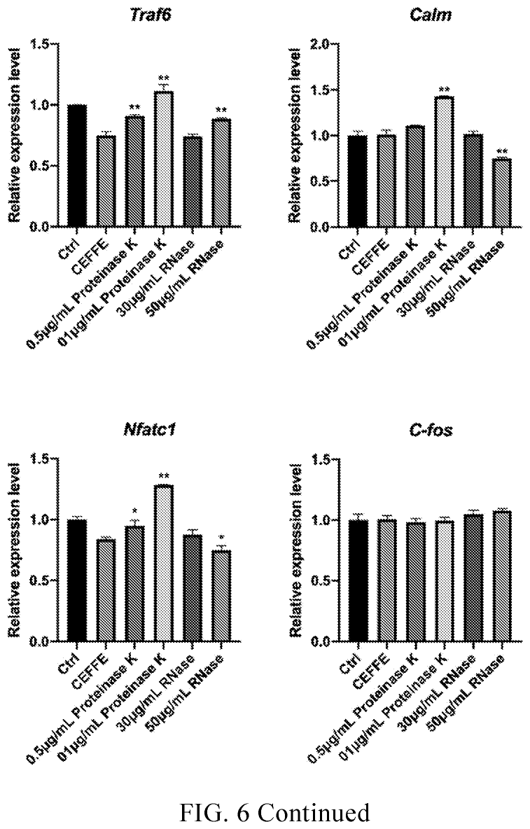

According to the rt-PCR results in FIG. 6, the inhibitory effect of CEFFE on osteoclast differentiation of BMM cells disappeared after CEFFE was digested with protease, while digestion of CEFFE with RNAase did not affect the inhibitory effect of CEFFE on osteoclast differentiation, thus indicating that the proteins in CEFFE are the main component that inhibit osteoblast differentiation and osteoclast differentiation of BMM.

4.2 The Cationic Component of CEFFE is the Main Component that Inhibit Osteoclast Differentiation of BMM

CEFFE was divided into anionic component, cationic component and strong cationic component by ion chromatography. BMM cells were inoculated in five 12-well plates and one 48-well plate, and divided into five groups after cell attachment, wherein 50 μg/mL of CEFFE, anionic component, cationic component and strong cationic component of CEFFE were added respectively. The medium containing CEFFE was changed every two days, and the mRNAs of each group of cells were extracted and stained with TRAP after six days.

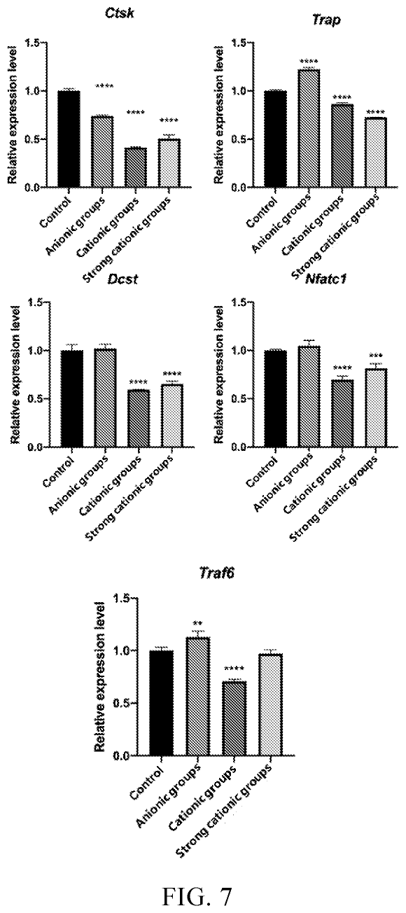

According to the results in FIG. 7, the cationic component and the strong cationic component of CEFFE could inhibit the osteoclast differentiation of BMM, while the anionic component could not significantly inhibit the osteoclast differentiation of BMM.

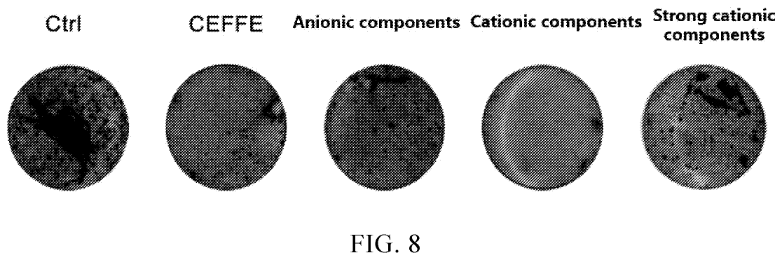

FIG. 8 showed the effect of different charge components contained in CEFFE on the osteoblast differentiation and osteoclast differentiation of BMM detected by TRAP staining. The cationic component and CEFFE can significantly inhibit the osteoclast differentiation of BMM, while the anionic component and strong cationic component do not inhibit the osteoclast differentiation of BMM as significantly as the cationic component, so the cationic component in CEFFE is the main component that inhibits the osteoclast differentiation of BMM.

Thus, the cationic component of CEFFE with appropriate ionic strength mainly inhibit osteoclast differentiation of BMM.

### Example 5. Therapeutic Effect of CEFFE on Bone Loss in the Hind Limbs of Tail Suspension Mice

The present invention used the mouse OVX model in vivo. After successful modeling, CEFFE (30 mg/kg) was injected into the tail vein, and 4 weeks later the material was taken for testing to evaluate the therapeutic effect of CEFFE on hind limb bone loss in tail suspension mice.

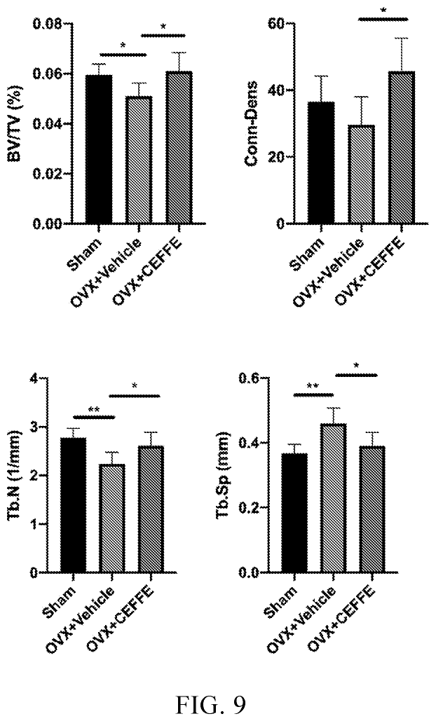

The results of micro CT analysis of cancellous and cortical bone of the tibia of the mice were shown in FIG. 9. CEFFE improved the structure of hind limb bone, alleviated bone loss and attenuated bone resorption: the bone volume fraction (BV/TV) of cancellous bone increased, the number of bone trabecula (Tb.N) increased, the connection density (Conn-Dens) increased, and the trabecular separation (Tb.sp) reduced in CEFFE group.

### Example 6

The present invention used the mouse OVX model in vivo. After successful modeling, CEFFE was injected into the tail vein, and 6 weeks later the material was taken for testing to evaluate the effect of CEFFE on bone mass of cancellous and cortical bone of the tibia and mechanical properties of the femur in mice.

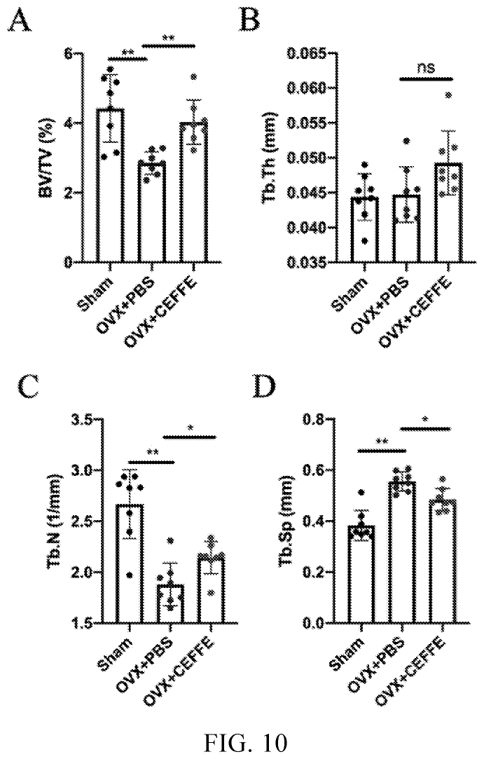

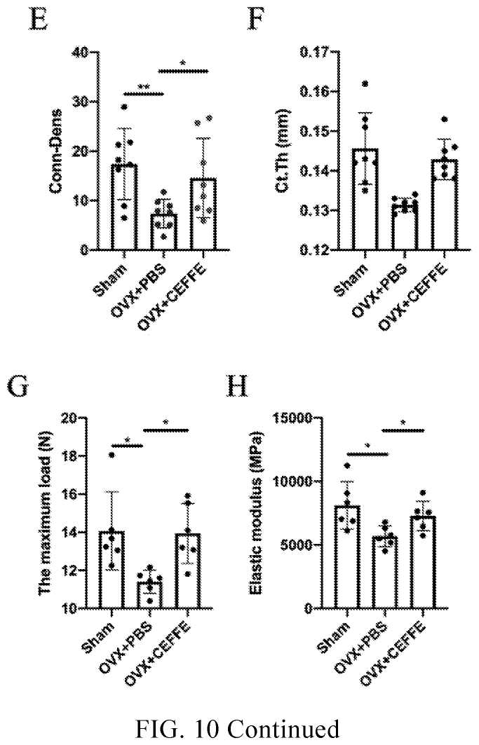

The results are shown in A-F in FIG. 10. The changes in tibial bone mass of mice in each group were analyzed by Micro CT, and it was found that compared with the PBS control group, tibial cancellous bone mass (BV/TV) increased (A), the number of bone trabecula (Tb.N) (C) and connection density (Conn. Dens) (E) increased, and the degree of trabecular separation (Tb. Sp) (D) reduced in mice after CEFFE administration for 6 weeks, and CEFFE can significantly increase the cortical bone thickness (Ct.Th) in OVX mice (F).

In addition, the maximum mechanical load and modulus of elasticity of the femur of OVX mice were reduced to some extent compared with that of normal mice detected by the three-point mechanical bending test, and the mechanical properties of the femur of OVX mice were significantly improved after the administration of CEFFE (G, H in FIG. 10).

### DISCUSSION

Tonnard first proposed the concept of nano-fat. Nano-fat containing lipid droplets, stromal vascular fractions (SVF) and growth factors can be obtained after mechanical emulsification of the fat obtained by liposuction. Nano-fat mainly works through SVF. SVF contains endothelial cells, adipose stem cells, macrophages, etc. On the one hand, cells can directly participate in tissue formation, and on the other hand, cells can promote tissue regeneration by secreting cytokines.

The difference from previous studies is that the present invention removes the living cells and lipid droplet component from nano-fat through centrifugation and filtration, does not contain stromal vascular fractions, and only retains growth factor components. Among them, there are various types of growth factors, including factors related to angiogenesis, inflammation, and estrogen. Experiments showed that injection of the cell-free fat extract of the present invention promoted bone quality improvement, improved bone mass and mechanical properties in ovariectomized mice, and inhibited the differentiation of BMM cells to osteoblast and osteoclast.

Compared with nano-fat, the cell-free fat extract has wider application prospects. Firstly, lipid droplet component is removed, and possible side effects are reduced; secondly, the cell component is removed, thus removing the immunogenicity, the application of allogeneic fat extract, mass production, and quality control can be realized in the future; thirdly, it is easy to cryopreserve and maintain biological activity, and no protective agent is required during cryopreservation, avoiding the contamination of other chemical composition.

All the documents cited herein are incorporated into the invention as reference, as if each of them is individually incorporated. Further, it would be understood that, in light of the above-described teaching of the invention, the skilled in the art could make various changes or modifications to the invention, and these equivalents are still in the scope of the invention defined by the appended claims of the application.

1. A method for preventing and/or treating osteoporosis, comprising the step of: administering a cell-free fat extract or a composition or preparation containing the cell-free fat extract to a subject in need thereof.

2. The method according to claim 1, wherein the osteoporosis is the osteoporosis caused by estrogenic decline.

3. The method according to claim 1, wherein the osteoporosis is postmenopausal osteoporosis.

4. The method according to claim 1, wherein the prevention and/or treatment of osteoporosis comprises one or more features selected from the group consisting of:
(i) inhibiting osteoblast differentiation and osteoclast differentiation;
(ii) inhibiting fusion of the BMM cytoskeleton;
(iii) increasing bone volume fraction;
(iv) increasing the number of bone trabecula;
(v) increasing bone connection density;
(vi) decreasing trabecular separation.

5. The method according to claim 1, wherein the cell-free fat extract contains one or more components selected from the group consisting of IGF-1, BDNF, GDNF, TGF-β1, HGF, bFGF, VEGF, TGF-β1, HGF, PDGF EGF, NT-3, GH, G-CSF, and the combinations thereof.

6. The method according to claim 1, wherein the dosage form of the composition or preparation is powder, granule, capsule, injection, tincture, oral solution, tablet or lozenge.

7. The method according to claim 1, wherein the injection is an intravenous injection or an intramuscular injection.

8. The method according to claim 1, wherein the cell-free fat extract is prepared by the following method comprising the following steps:
(1) providing a fat tissue raw material, shredding the fat tissue raw material, and rinsing (e.g., with physiological saline), thereby obtaining a rinsed fat tissue;
(2) centrifuging the rinsed fat tissue to obtain a layered mixture;
(3) discharging the excess liquid at the bottom and the grease on top from the layered mixture and collecting the intermediate layer (that is, the fat layer containing fat cells);
(4) subjecting the intermediate layer to mechanical emulsification to obtain a mechanically emulsified fat mixture (also called nano-fat);
(5) centrifuging the mechanically emulsified fat mixture (combined or not combined) to obtain a transparent (or substantially transparent) intermediate liquid layer, which is a primary fat extract; and
(6) subjecting the primary fat extract to filtration and sterilization to obtain the cell-free fat extract.

9. A method for preventing and/or treating osteoporosis, comprising the step of: administering proteins in a cell-free fat extract or a composition or preparation containing the proteins in the cell-free fat extract to a subject in need thereof,
wherein the proteins in the cell-free fat extract is a cell-free fat extract after RNA enzyme digestion or extracted therefrom.

10. A method for preventing and/or treating osteoporosis, comprising the step of: administering the cationic component in a cell-free fat extract or a composition or preparation containing the cationic component in the cell-free fat extract to a subject in need thereof;
wherein, the cationic component in the cell-free fat extract is the cationic component obtained by ion chromatography of the cell-free fat extract, or further extracted therefrom.

11. The method according to claim 1, wherein the cell-free fat extract is derived from adipose tissue.

12. The method according to claim 1, wherein the cell-free fat extract contains no cell and no lipid droplet, wherein the lipid droplets are oil droplets released after fat cells are disrupted.

13. The method according to claim 12, wherein the expression of “contain no lipid droplet” means that in the cell-free fat extract, the volume of oil droplets accounts for less than 1% of the total liquid.

14. The method according to claim 12, wherein the expression of “contain no cell” means that the average number of cells in 1 mL of the cell-free fat extract is ≤1.

15. The method according to claim 1, wherein the cell-free fat extract is a naturally-obtained nano-fat extract without added ingredients.

16. The method according to claim 15, wherein the expression of “without added ingredients” means that no solution, solvent, small molecule, chemical preparation, and biological additive are added during the preparation of the fat extract except rinsing step.
